(19) United States
(12) Patent Application Publication
(10) Pub. No.: US 2012/0301363 A1
(43) Pub. Date: Nov. 29, 2012

(54) MIX-TYPE CATALYST FILTER AND MANUFACTURING METHOD THEREOF

(75) Inventors: Jee Yong KIM, Seoul (KR); Rae Eun PARK, Seongnam-si (KR); Jeong Myeong KIM, Seoul (KR)

(73) Assignee: SAMSUNG ELECTRONICS CO., LTD., Suwon-si (KR)

(21) Appl. No.: 13/477,572

(22) Filed: May 22, 2012

(30) Foreign Application Priority Data
May 26, 2011 (KR) 10-2011-0049952

(51) Int. Cl. B01J 23/86; B01J 27/04; B01J 21/06; B01J 23/06; B01J 23/30; B01J 31/06; A61L 9/00; B01J 37/08; B01J 23/14; B82Y 30/00; B82Y 40/00

(52) U.S. Cl. 422/122; 502/100; 502/216; 502/350; 502/343; 502/352; 502/305; 502/349; 502/316; 502/159; 502/300; 977/900; 977/762

(57) ABSTRACT

A mix-type catalyst filter which has a variety of pore sizes and thus improves efficiency of catalysts and a method for manufacturing the same. The method includes spinning nanofibers, heating the nanofibers, crushing the nanofibers to form chip-type nanofibers, mixing the chip-type nanofibers with particulate catalysts to obtain a mix-type catalyst and heating the mix-type catalyst.

### CROSS-REFERENCE TO RELATED APPLICATIONS

This application claims the benefit of Korean Patent Application No. 10-2011-0049952, filed on May 26, 2011 in the Korean Intellectual Property Office, the disclosure of which is incorporated herein by reference.

### BACKGROUND

1. Field

Embodiments of the present invention relate to a mix-type catalyst filter to improve efficiency of catalysts and a method for manufacturing the same.

2. Description of the Related Art

Catalysts remove and degrade contaminants present in air or water. At present, methods for purifying air using photocatalysts are suggested. A photocatalyst refers to one type of semiconductor ceramic which reacts with light and thus serves as a catalyst.

Titanium dioxide (TiO₂) is used as a representative photocatalyst. Titanium dioxide absorbs ultraviolet light when light is irradiated to produce electrons and holes. These electrons and holes have a strong reducing force and a strong oxidizing force, respectively. In particular, holes react with water and dissolved oxygen and the like to produce OH radicals and active oxygen. As a result, OH radical energy is higher than the bonding energy of molecules constituting organics, thus enabling degradation through simple severing. For this reason, titanium dioxide is used for environmental clean-up of a variety of substances contained in air, including toxic chemical substances and malodorous substances, as well as degradation and toxicity-removal of these substances and degradation of contaminants.

However, these catalysts are provided in the form of particles and thus have a limited size, thus disadvantageously causing great differences in efficiency depending on targets to be removed.

Also, a rate of catalysts diffused into a catalyst layer is excessively low due to excessively small pore sizes and rapid degradation of malodorous substances is thus disadvantageously impossible.

### SUMMARY

Therefore, it is one aspect of the present disclosure to provide a method for manufacturing a mix-type catalyst filter which has different pore sizes and can thus absorb a variety of gases and a mix-type catalyst filter manufactured by the method.

Also, it is another aspect of the present disclosure to provide a method for manufacturing a mix-type catalyst filter in which nanofibers, particulate catalysts and chip-type catalysts (hereinafter, referred to as a “mix-type catalyst”) are mixed and the distribution of inner pores is random and diversified, and a mix-type catalyst filter manufactured by the method.

Additional aspects will be set forth in part in the description which follows and, in part, will be obvious from the description, or may be learned by practice of the invention.

In accordance with one aspect, a method for manufacturing a mix-type catalyst filter includes: spinning nanofibers; heating the nanofibers; crushing the nanofibers to form chip-type nanofibers; mixing the chip-type nanofibers with particulate catalysts to obtain a mix-type catalyst; and heating the mix-type catalyst.

The method may further include coating the mix-type catalyst on a filter support.

The particulate catalysts may be TiO₂, ZnO, SnO₂, WO₃, ZrO₂ or CdS.

The method may further include heating the mix-type catalyst to remove impurities and activate the particulate catalysts after heating.

The nanofibers may be spun by solution spinning or melt spinning.

The filter support may be a substance to support the nanofiber, selected from a porous substrate, stainless steel, a glass plate, a metal, a ceramic, an organic polymer and wood.

The size of the particulate catalysts may be increased by lengthening heating time.

The size of the particulate catalysts is decreased by shortening heating time.

The particulate catalysts may have different sizes, and particulate catalysts with a larger particle size may be arranged towards the outside from the surface of the nanofibers.

In accordance with another aspect, a method for manufacturing a mix-type catalyst filter includes: spinning nanofibers on a filter support; permeating the nanofibers into the filter support; coating the nanofibers permeated into the filter support with particulate catalysts to obtain a mix-type catalyst; and heating the mix-type catalyst.

The permeation of the nanofibers into the filter support may be carried out using a water jet or an air jet.

The size of the particulate catalysts may be controlled by controlling heating time.

The particulate catalysts may have different sizes and particulate catalysts with a larger particle size may be arranged towards the outside from the surface of the nanofibers.

In accordance with another aspect, a mix-type catalyst filter includes: nanofibers; and particulate catalysts having different sizes adsorbed onto the nanofibers.

The nanofibers may be monofibers or chip-type catalysts.

The size of the particulate catalysts may be controlled by controlling heating time.

The particulate catalysts may have different sizes and particulate catalysts with a larger particle size may be arranged towards the outside from the surface of the nanofibers.

In accordance with another aspect, a mix-type catalyst filter includes: nanofibers; and particulate catalysts having different sizes adsorbed onto the nanofibers, wherein the particulate catalysts are TiO₂.

The size of the particulate catalysts may be controlled by controlling heating time and particulate catalysts with a larger particle size may be arranged such that particulate catalysts with a larger particle size are dispersed towards the outside from the surface of the nanofibers.

The particulate catalysts may be prepared without separate binding.

In accordance with another aspect of the present invention, an air conditioner includes: a body provided with at least one inlet; a ventilator provided in the body to intake exterior air; and a mixed-type catalyst filter containing nanofibers and particulate catalysts having different sizes adsorbed onto the nanofibers to purify air supplied through the ventilator.

The particulate catalysts may be arranged such that particulate catalysts with a larger particle size are dispersed towards the outside from the surface of the nanofibers.

### BRIEF DESCRIPTION OF THE DRAWINGS

These and/or other aspects of the invention will become apparent and more readily appreciated from the following description of the embodiments, taken in conjunction with the accompanying drawings of which:

FIG. 1 is a schematic view illustrating a mix-type catalyst filter according to one embodiment of the present invention;

FIG. 2 is an enlarged view of part “A” of FIG. 1;

FIGS. 3A to 3F are schematic views illustrating a method for manufacturing a mix-type catalyst filter according to an embodiment of the present invention;

FIGS. 4A to 4E are schematic views illustrating a method for manufacturing a mix-type catalyst filter according to an embodiment of the present invention;

FIG. 5 is a schematic view illustrating a mix-type catalyst filter according to an embodiment of the present invention;

FIG. 6 is a schematic view illustrating a mix-type catalyst filter according to an embodiment of the present invention;

FIGS. 7A to 7D are schematic views illustrating a method for manufacturing a mix-type catalyst filter according to an embodiment of the present invention; and

FIG. 8 is a schematic view illustrating an air conditioner provided with a mix-type catalyst filter according to an embodiment of the present invention.

### DETAILED DESCRIPTION

Reference will now be made in detail to the embodiments of the present invention, examples of which are illustrated in the accompanying drawings, wherein like reference numerals refer to the like elements throughout.

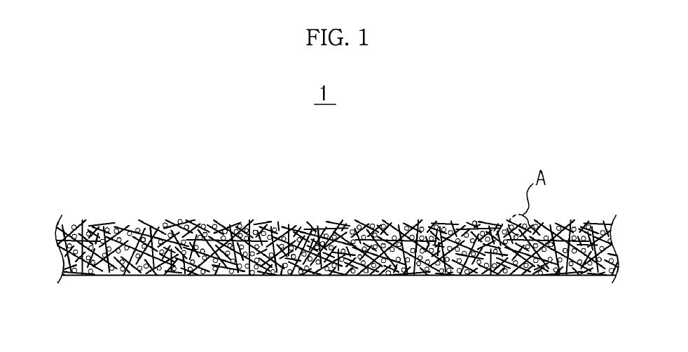

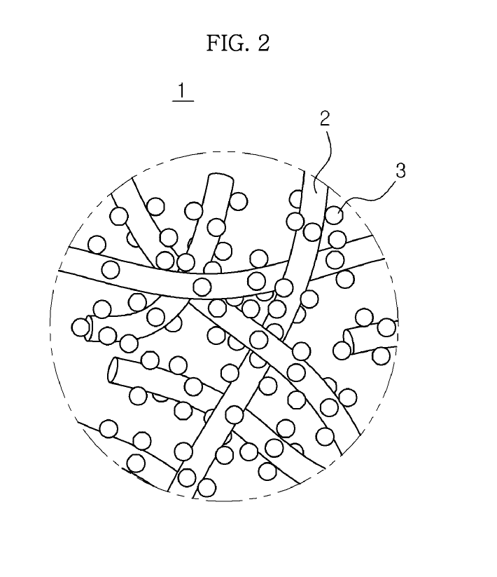

As shown in FIGS. 1 to 2, a mix-type catalyst 1 includes a plurality of nanofibers 2 and a plurality of particulate catalysts 3.

The nanofibers 2 are microfibers which have a diameter of several tens to hundreds of nanometers (nm) and are used as filters owing to their large surface area per unit volume.

However, a structural layer including only the nanofibers 2 has greatly decreased light-transmittance due to considerably high filter resistance and large diameter, thus disadvantageously causing deterioration in photo-dissociation efficiency.

The particulate catalysts 3 are photocatalyst semiconductors and examples thereof include TiO₂, ZnO, SnO₂, WO₃, ZrO₂, CdS and the like. Of these, titanium dioxide (Titan, TiO₂, anatase-type) is used.

Titanium dioxide photocatalysts are n-type semiconductors which absorb ultraviolet light, generating electrons and holes, when light is irradiated thereto. These electrons and holes have strong reducing and oxidizing forces, respectively.

In particular, holes react with water, dissolved oxygen and the like to produce OH radicals and active oxygen. As a result, OH radical energy is higher than biding energy of molecules constituting organics and enables degradation through easy cleaving. Thus being applicable to harmful chemicals and malodorous substances contained in the air as well as various environmental remediation fields including degradation and removal of harmfulness of chemical substances in the air and degradation of contaminants.

Based on this principle, contaminants in the air are decomposed and are thus converted into harmless water and carbonic acid gas. Titanium dioxide photocatalysts are often called “semiconductor photocatalysts”, since they use n-type semiconductor functions.

However, the particulate catalysts 3 have a diameter of several tens to hundreds of nanometers and thus excessively small crystal size and small pore size. Therefore, the particulate catalysts 3 exhibits a low rate of gas diffusion into the catalyst layer. Also, the particulate catalysts 3 disadvantageously have a difference in efficiency depending on target substances to be removed due to small pore size.

Accordingly, an embodiment provides a mix-type catalyst filter 1′ which contains a mix-type catalyst 1 in which the nanofibers 2 are mixed with the particulate catalysts 3 to provide a variety of pore sizes and a method for manufacturing the same.

The mix-type catalyst 1 has a structure in which the particulate catalysts 3 are adsorbed onto the surface of the nanofibers 2. By mixing the nanofibers 2 having a crystal size of several tens to several hundreds of nanometers with the particulate catalysts 3 having a crystal size of several to several tens of nanometers, a variety of pore sizes can be realized and various contaminant gases can thus be removed.

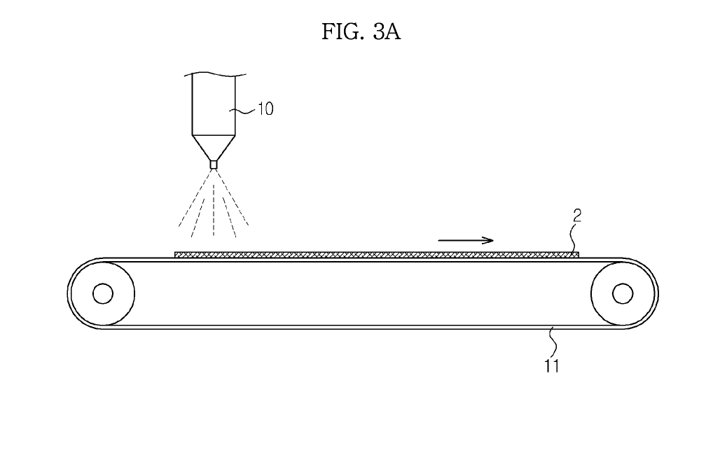

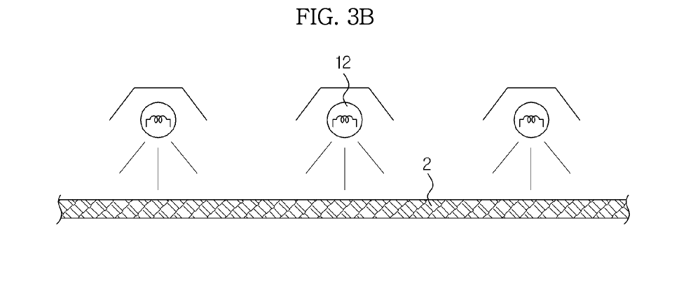

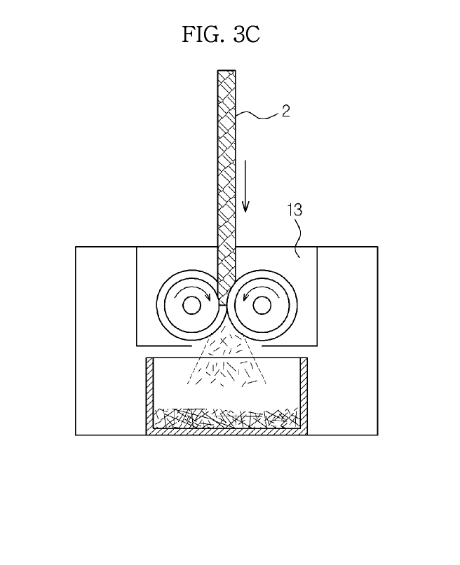

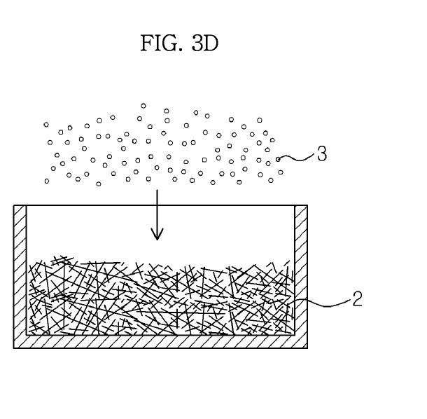

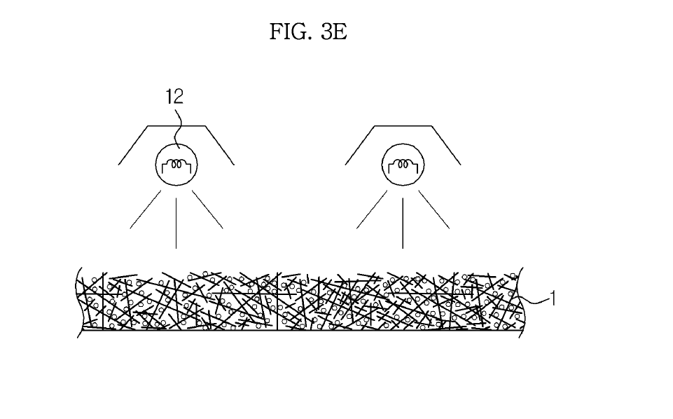

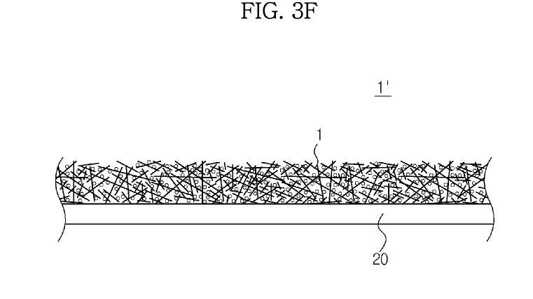

As shown in FIGS. 3A to 3F, the method for manufacturing the mix-type catalyst 1 according to an embodiment includes 1) spinning nanofibers, 2) heating the nanofibers, 3) crushing the nanofibers to form chip-type nanofibers, 4) mixing the chip-type nanofibers with particulate catalysts to obtain a mix-type catalyst, and 5) heating the mix-type catalyst.

First, the nanofibers 2 is spun on a conveyer belt 11 to prevent dispersion of the nanofibers 2 (FIG. 3A).

At this time, a spinner 10 is preferably a solution spinner or melt spinner.

The spun nanofibers 2 are heated in a heater 12 and crushed into a chip shape in a crushing machine 13 (FIGS. 3B and 3C). The heated nanofibers 2 are dehydrated and are thus converted into dried chips. The chip-type crushed nanofibers 2 are mixed with particulate catalysts 3 and heated to form a mix-type catalyst 1.

The mix-type catalyst 1 thus formed is coated on a filter support 20 to form a mix-type catalyst filter 1′.

At this time, the filter support 20 is preferably a substance to support the nanofibers 2 and preferred examples thereof include porous substrates, stainless steel, glass plates, metals, ceramics, organic polymers and wood.

Also, the method may further include heating the mix-type catalyst 1 obtained after the heating process to remove impurities present in the mix-type catalyst 1 and activate the particulate catalysts 3.

After heating, the inside of the particulate catalysts 3 may be converted into an anatase structure and the outside thereof may be converted into a rutile or brookite structure.

Anatase and rutile particulate catalysts are capable of serving as photocatalysts, although they depend on crystalline structure and index of refraction.

Anatase belongs to the tragonal system and is a sharp awl-shaped crystal and is sometimes a flat plate-shaped crystal.

In addition, rutile belongs to the tragonal system and is a rod- or needle-shaped crystal.

In addition, brookite is a flat plate-shaped, rarely, awl-shaped crystal.

The particulate catalysts 3 are preferably using in combination of anatase and another structure such as rutile rather than when using anatase alone.

The particulate catalyst, that is, titanium dioxide is cleaved into electrons and holes when light is irradiated thereto. The electrons separated from the holes are bonded to holes again. At this time, as rebonding is further delayed, the amount of OH radicals increases. The reason for this is that, since OH radicals are produced from holes, when the electron holes are occupied by the electrons again, production of OH radicals is stopped.

As such, when the rutile structure contacts the anatase structure, the rutile structure receives holes separated from the anatase holes, thus lengthening the presence time of the anatase holes.

Accordingly, as the amount of OH radicals increases, degradation of various chemical substances including harmful chemical substances and malodorous substances and of contaminants contained in the air is activated. As a result, the particulate catalysts 3 exhibit about 20% higher efficiency, when composed of a combination of anatase and other structure such as rutile than when composed of 100% of anatase.

The size (diameter) of the particulate catalyst 3 can be controlled by adjusting the heating time of the heater 12.

That is, the size of the particulate catalysts 3 can be decreased by shortening the heating time and, conversely, the size of the particulate catalysts 3 can be increased by lengthening the heating time.

At this time, when the heating time of the particulate catalysts 3 increases, recombination between particles occurs, thus increasing the size thereof.

The size of the particulate catalyst 3 is measured at a temperature of 400° C. for different times of 1, 2 and 4 hours and the results thus obtained are shown in the following Table 1.

TABLE 1

| Time | Size of particle | Heating temperature (° C.) |
|---|---|---|
| 1 hr | 5 nm | 400° C. |
| 2 hr | 8 nm | |
| 4 hr | 25 nm | |

As can be seen from Table 1 above, the particulate catalysts 3 may have different sizes. By mixing the particulate catalysts 3 having different sizes with the nanofibers 2, the mix-type catalyst 1 can also be formed.

When the particulate catalysts 3 are adsorbed onto the surface of the nanofibers 2, particulate catalysts 3 with different sizes are arranged such that particulate catalysts 3 with a larger particle size are dispersed towards the outside from the surface of the nanofibers 2.

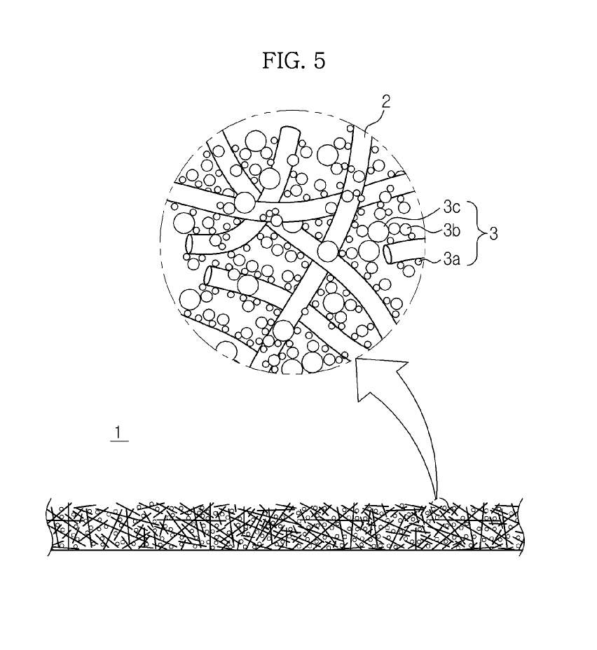

For example, the particulate catalysts 3 have different sizes such as 5 nm (3a), 8 nm (3b) and 25 nm (3c), and 5 nm (3a), 8 nm (3b) and 25 nm (3c) particulate catalysts are arranged in this order towards the outside from the surface of the nanofibers 2 (FIG. 5).

When the particulate catalysts 3 having different sizes form the mix-type catalyst 1 with the nanofibers 2, sizes of the pores can be diversified. Therefore, a variety of gases can thus be removed, a rate of the odor diffused into the catalyst layer can be improved and purification efficiency can thus be improved.

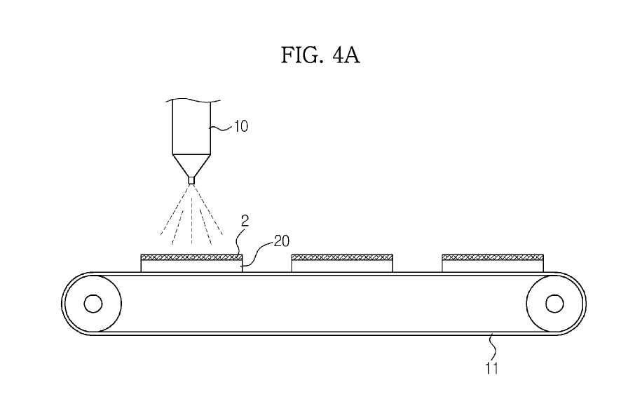

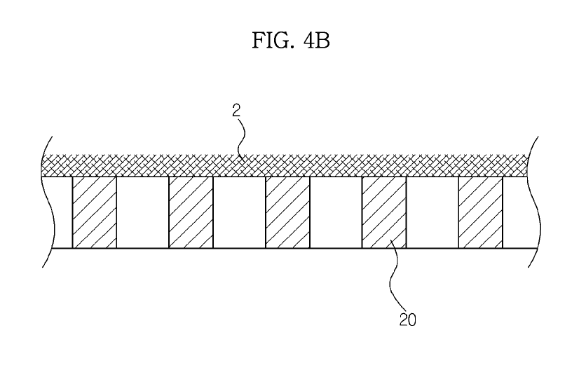

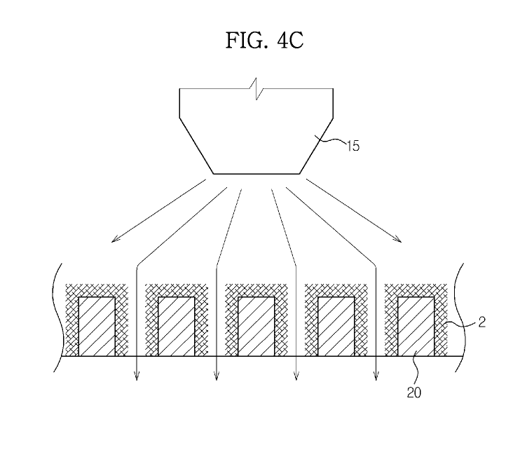

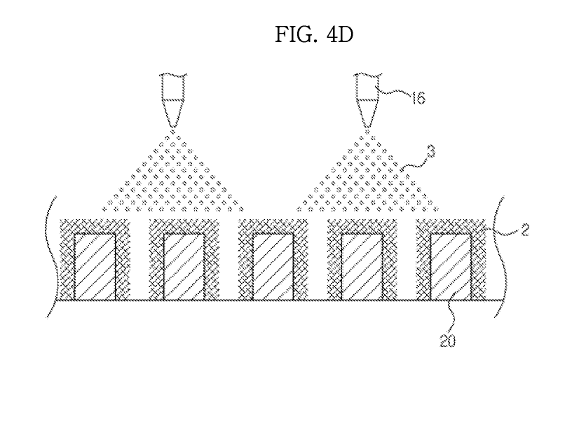

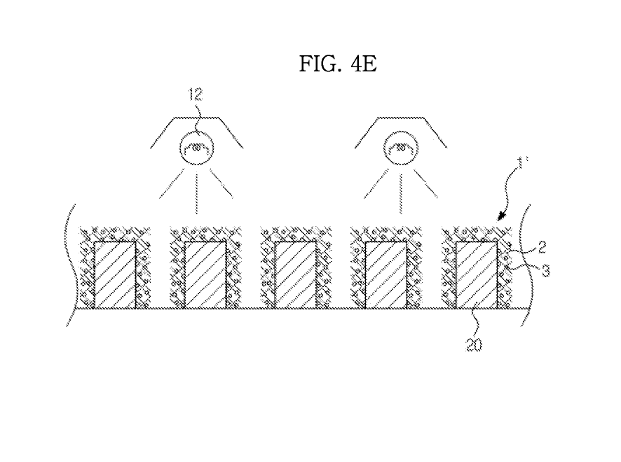

As shown in FIGS. 4A to 4E, the method for manufacturing the mix-type catalyst filter 1′ according to an embodiment of the present invention includes 1) spinning nanofibers on a filter support, 2) permeating the nanofibers into the filter support, 3) coating the nanofibers permeated into the filter support with particulate catalysts to obtain a mix-type catalyst and 4) heating the mix-type catalyst.

The filter support 20 is preferably a substance to support the nanofibers 2 without being dispersed and preferred examples thereof include porous substrates, stainless steel, glass plates, metals, ceramics, organic polymers and wood.

The spun nanofibers 2 are permeated into the filter support 20 and then coated with particulate catalysts 3 to obtain a mix-type catalyst 1 (FIG. 4D).

The permeation of the nanofibers 2 into the filter support 20 is preferably carried out using a water jet 15 or an air jet, or by air blowing.

Water or air is sprayed at a high pressure onto the spun nanofibers 2 using the water jet or air jet to sever the nanofibers 2 and allow the nanofibers 2 to be permeated into the filter support 20 (FIG. 4C).

At this time, the nanofibers 2 are preferably monofibers or chip-type catalysts.

The mix-type catalyst containing the filter support 20 thus obtained, the nanofibers 2 and the particulate catalyst 3 are heated using a heater 12 to activate the particulate catalysts 3 (FIG. 4E).

At this time, the size (diameter) of the particulate catalysts 3 can be controlled by controlling heating time of the particulate catalysts 3. The variation in the size of the particulate catalyst 3 is the same as described in Table 1 and a description associated with the embodiment in conjunction with the same and a detailed explanation thereof is thus omitted.

Accordingly, as shown in FIG. 5, a variety of pore sizes can be formed, a variety of gases can be removed and purification efficiency can thus be improved by using the particulate catalysts 3 with a variety of sizes for the preparation of mix-type catalyst filters.

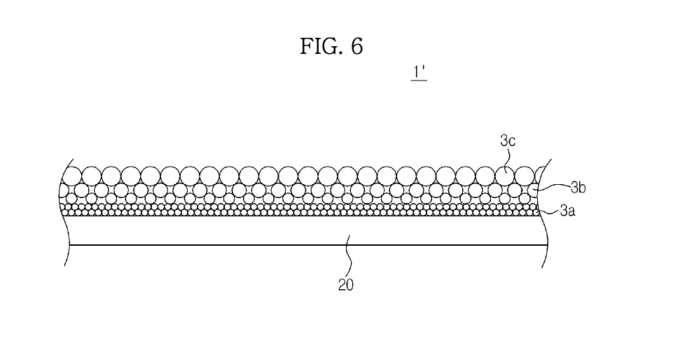

As shown in FIG. 6, the mix-type catalyst filter 1′ according to an embodiment of the present invention includes a filter support 20 and a plurality of particulate catalysts 3 with a variety of sizes adsorbed onto the filter support 20.

The particulate catalyst 3 is preferably TiO₂, ZnO, SnO₂, WO₃, ZrO₂, or CdS and is generally TiO₂.

At this time, the size (diameter) of the particulate catalysts 3 can be controlled by controlling heating time of the particulate catalysts 3. Variation in the size of the particulate catalysts 3 is the same as described in Table 1 and a description associated with the embodiment in conjunction with the same and a detailed explanation thereof is thus omitted.

The particulate catalysts 3 adsorbed onto the surface of the nanofibers 2 are arranged such that particulate catalysts 3 with a larger particle size are dispersed towards the outside from the surface of the nanofibers 2.

For example, the particulate catalysts 3 have different sizes such as 5 nm (3a), 8 nm (3b) and 25 nm (3c), and the 5 nm (3a), 8 nm (3b) and 25 nm (3c) particulate catalysts are arranged in this order towards the outside from the surface of the nanofibers 2.

Accordingly, the particulate catalysts 3 on a nanometer (nm) scale are arranged in the particulate catalyst layer to improve adsorption.

Also, pores with a variety of sizes are formed in this order and a variety of types of gases can thus be removed.

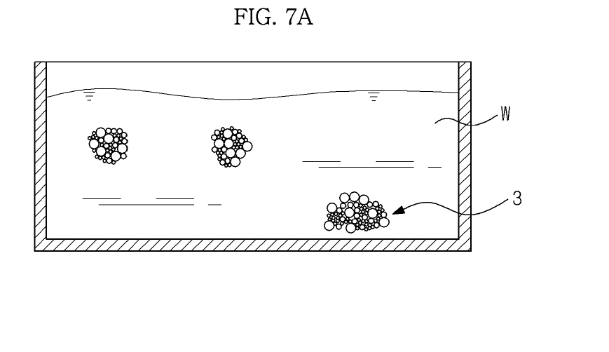

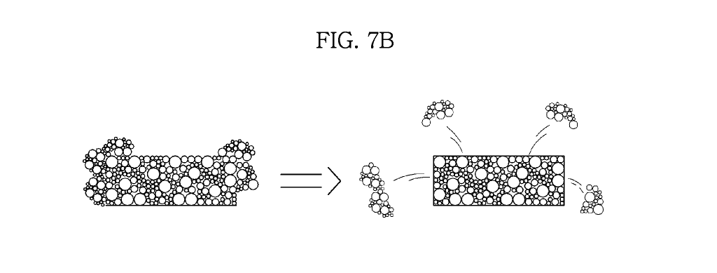

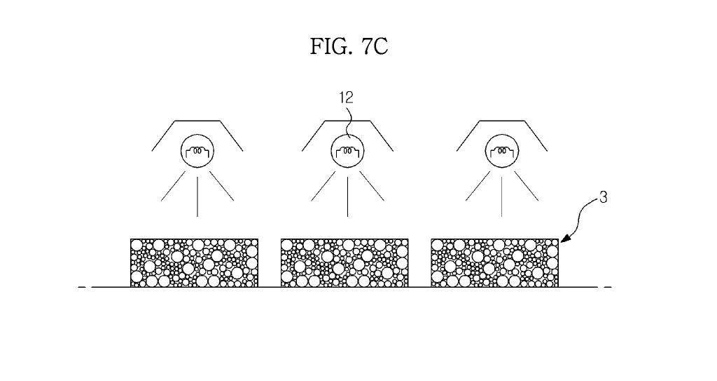

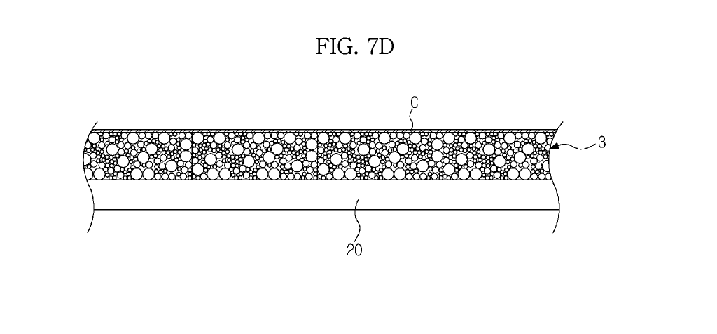

As shown in FIGS. 7A to 7D, the method for manufacturing the mix-type catalyst filter 1′ according to an embodiment of the present invention includes 1) dispersing particulate catalysts with a variety of sizes in water to aggregate the particulate catalysts through attractive force and 2) heating the aggregated particulate catalysts, crushing the catalysts and then coating the same on a filter support.

The dispersed particulate catalysts 3 (3a, 3b, 3c, hereinafter, represented by reference numeral “3”) are aggregated through attractive force in a solvent or pure water W causing no variation in pH.

The particulate catalysts 3 are aggregated by a precipitation, immersion, hydrothermal synthesis, sol-gel, plasma or spray method and are applied to the filter support 20 using at least two process conditions and times.

The aggregated particulate catalysts 3 have a variety of shapes which are not specifically defined (FIG. 7A).

The aggregated particulate catalysts 3 are heated in a heater 12 and crushed to a predetermined size.

At this time, the crushed particulate catalysts 3 have identical volumes, but different shapes. The reason for this is that the aggregation of the particulate catalysts 3 is not carried out in accordance with a specific rule.

The particulate catalysts 3 crushed to a specific size are coated onto the filter support 20.

Accordingly, the particulate catalysts 3 having different sizes are adsorbed in various forms on the filter support 20 and a variety of gases can be removed through a variety of pore sizes.

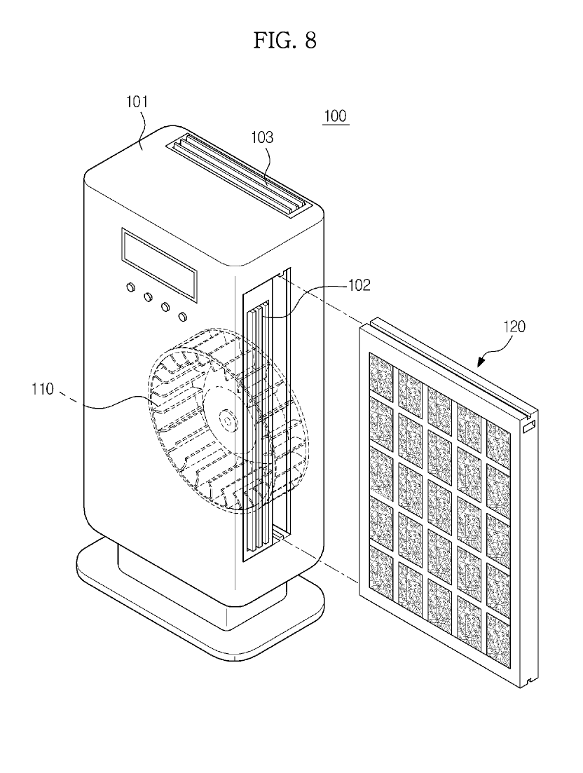

As shown in FIG. 8, an air conditioner 100 according to one embodiment of the present invention includes a body 101 provided with an inlet 102, a ventilator 110 provided in the body 101 and a filter 120 to purify air supplied through the ventilator 110.

The inlet 102 is provided in the body 101 to intake indoor air. The body 101 is provided at the top thereof with an outlet 103 to discharge air passing through the body 101 indoors.

The filter 120 is provided inside the inlet 102 to filter out foreign materials present in air absorbed in the body 101.

The filter 120 is manufactured by the manufacturing method thereof using the mix-type catalyst filter 1′ which contains a mixture of the nanofibers 2 and the particulate catalysts 3 such that a variety of pore sizes of this embodiment are imparted to the mix-type catalyst filter 1′.

The mix-type catalyst filter 1′ and the method for manufacturing the same are the same as in the previous embodiments and a detailed description thereof is thus omitted.

Accordingly, the air conditioner provided with the mix-type catalyst filter 1′ can remove a variety of gases through the mix-type catalyst filter 1′ having a variety of pore sizes and thus exhibits superior air clearance effects.

According to the embodiments of the present disclosure, a variety of gases can be absorbed and superior purification efficiency can thus be obtained by forming a variety of pore sizes.

Also, gas diffusion rate and gas adsorption rate are improved and deordorization performance can thus be improved through such a variety of pore sizes.

Although a few embodiments of the present disclosure have been shown and described, it would be appreciated by those skilled in the art that changes may be made in these embodiments without departing from the principles and spirit of the invention, the scope of which is defined in the claims and their equivalents.

What is claimed is:

1. A method for manufacturing a mix-type catalyst filter comprising:
spinning nanofibers;
heating the nanofibers;
crushing the nanofibers to form chip-type nanofibers;
mixing the chip-type nanofibers with particulate catalysts to obtain a mix-type catalyst; and
heating the mix-type catalyst.

2. The method according to claim 1, further comprising:
coating the mix-type catalyst on a filter support.

3. The method according to claim 1, wherein the particulate catalysts are TiO₂, ZnO, SnO₂, WO₃, ZrO₂ or CdS.

4. The method according to claim 1, further comprising:
heating the heated mix-type catalyst to remove impurities and activate the particulate catalysts after the heating process.

5. The method according to claim 1, wherein the nanofibers are spun by solution spinning or melt spinning.

6. The method according to claim 2, wherein the filter support is a substance to support the nanofibers, selected from a porous substrate, stainless steel, a glass plate, a metal, a ceramic, an organic polymer and wood.

7. The method according to claim 1, wherein the size of the particulate catalysts is increased by lengthening heating time.

8. The method according to claim 1, wherein the size of the particulate catalysts is decreased by shortening heating time.

9. The method according to claim 7 or 8, wherein the particulate catalysts have different sizes and wherein particulate catalysts with a larger particle size are arranged towards the outside from the surface of the nanofibers.

10. A method for manufacturing a mix-type catalyst filter, comprising:
spinning nanofibers on a filter support;
permeating the nanofibers into the filter support;
coating the nanofibers permeated into the filter support with particulate catalysts to obtain a mix-type catalyst; and
heating the mix-type catalyst.

11. The method according to claim 10, wherein the permeation of the nanofibers into the filter support is carried out using a water jet or an air jet.

12. The method according to claim 10, wherein the size of the particulate catalysts is controlled by controlling heating time.

13. The method according to claim 12, wherein the particulate catalysts have different sizes and wherein particulate catalysts with a larger particle size are arranged towards the outside from the surface of the nanofibers.

14. A mix-type catalyst filter comprising:
nanofibers; and
particulate catalysts having different sizes adsorbed on the nanofibers.

15. The filter according to claim 14, wherein the nanofibers are monofibers or chip-type catalysts.

16. The filter according to claim 14, wherein the size of the particulate catalysts is controlled by controlling heating time.

17. The filter according to claim 15, wherein the particulate catalysts have different sizes and wherein particulate catalysts with a larger particle size are arranged towards the outside from the surface of the nanofibers.

18. A mix-type catalyst filter comprising:
nanofibers; and
particulate catalysts having different sizes adsorbed onto the nanofibers,
wherein the particulate catalysts are TiO₂.

19. The filter according to claim 18, wherein the size of the particulate catalysts is controlled by controlling heating time and particulate catalysts with a larger particle size are arranged such that the particulate catalysts with a larger particle size are dispersed towards the outside from the surface of the nanofibers.

20. The filter according to claim 18, wherein the particulate catalysts are prepared without separate binding.

21. An air conditioner comprising:
a body provided with at least one inlet;
a ventilator provided in the body to intake indoor air; and
a mixed-type catalyst filter comprising nanofibers and particulate catalysts having different sizes adsorbed onto the nanofibers to purify air supplied through the ventilator.

22. The air conditioner according to claim 21, wherein the particulate catalysts are arranged such that the particulate catalysts with a larger particle size are dispersed towards the outside from the surface of the nanofibers.

23. The method according to claim 1, wherein the particular catalysts are aggregated through attractive force in a solvent or pure water.

24. The method according to claim 23, wherein the particular catalysts are aggregated by at least one of precipitation, immersion, hydrothermal synthesis, sol-gel plasma or spray method.

25. The method according to claim 2, wherein the particular catalyst are applied to the filter support using at least two process conditions and times.

26. The method according to claim 10, wherein the particular catalysts are aggregated through attractive force in a solvent or pure water.

27. The method according to claim 26, wherein the particular catalysts are aggregated by at least one of precipitation, immersion, hydrothermal synthesis, sol-gel plasma or spray method.

28. The method according to claim 10, wherein the particular catalyst are applied to the filter support using at least two process conditions and times.
